(19) United States
(12) Patent Application Publication
(10) Pub. No.: US 2023/0101520 A1
(43) Pub. Date: Mar. 30, 2023

(54) Integrated Bioreactor Apparatus for Fabricating Polyhydroxyalkanoate Bioplastic Products

(71) Applicant: Nouf Ibrahim Alhazmi, Benicia, CA (US)

(72) Inventor: Nouf Ibrahim Alhazmi, Benicia, CA (US)

(21) Appl. No.: 17/449,281

(22) Filed: Sep. 29, 2021

(51) Int. Cl. C12P 7/625; C12M 1/12; C12M 3/00; C12M 1/06; C12M 1/00; C12M 1/34; C12M 1/36

(52) U.S. Cl. CPC C12P 7/625; C12M 23/06; C12M 23/44; C12M 27/02; C12M 29/04; C12M 37/00; C12M 41/26; C12M 41/34; C12M 41/32; C12M 41/48; C12M 41/12

(57) ABSTRACT

The present disclosure provides an integrated and energy-efficient apparatus for the fabrication of completely biodegradable Polyhydroxyalkanoate, PHA, bioplastic products. The apparatus combines a bioreactor fermenter, mixing chambers, and an oven and mold chamber in a single compact design, allowing the entire fabrication process to be handled by a process controller if desired. Plates, cups, masks, packaging materials, and other plastic products can be produced directly from biomass media, with solvents and other agents recycled within the apparatus between batches.

### FIELD OF INVENTION

The present invention relates generally to an end-to-end fabrication process for bioplastics. More specifically, the present invention relates to an integrated fermentation apparatus for fabricating biodegradable products from Polyhydroxyalkanoates (PHA).

### BACKGROUND

It is well documented that the negative impact on the environment from the production of conventional plastics is enormous. The problem of plastic pollution has only been further exacerbated by the COVID-19 pandemic, with the mass production of disposable protective gear such as face coverings that are partially made of polyethylene and other types of non-biodegradable plastics.

As a result, more and more interest is being drawn towards the production of biodegradable bioplastics, plastic materials produced from renewable biomass sources, such as organic industrial waste, recycled food waste, etc. However, the majority of bioplastics available on the market today are made directly from plants, and thus compete with food industry for source materials. Furthermore, few bioplastics are available on an industrial scale, and many are not even fully biodegradable, instead requiring special processing facilities to decompose.

The same is true for so-called “compostable plastics” that are derived from petroleum-based plastics. For example, the compostable plastic available on the market under the label “PLA” requires complicated processing facilities to degrade (it is not technically biodegradable, only degradable) because there must be high humidity and temperature for the degrading to happen.

Polyhydroxyalkanoate, PHA, is a bioplastic which is gaining popularity due to being the single truly biodegradable bioplastic, as it will degrade in the same conditions as natural biomass such as fruit. PHA is currently not widely available at an industrial scale, and there are only a handful of companies that produce it for non-biomedical applications. This is partially due to the fact that no adequately efficient processes or apparatus for manufacturing it at scale have been developed.

There are research papers and theoretical documents which discuss the possibility of extracting bioplastics such as PHA from bacteria, but it has yet to be put into practice in a manner which can scale. In current practices, scientists typically perform the fermentation of the necessary PHA biomass in one or more bioreactors, flow reactor, or flask, then move it to another flask to mix with an agent and add solvents. After multiple steps of filtration, extraction, and purification, the biomass is dried for a day or so at room temperature. Finally, the dried PHA granules or resins are used to make an actual product such as a plastic sheet or plate.

It is within this context that the present invention is provided.

### SUMMARY

The present disclosure provides an integrated and energy-efficient apparatus for the fabrication of completely biodegradable Polyhydroxyalkanoate, PHA, bioplastic products. The apparatus combines a bioreactor fermenter, mixing chambers, and an oven and mold chamber in a single compact design, allowing the entire fabrication process to be handled by a process controller if desired. Plates, cups, masks, packaging materials, and other plastic products can be produced directly from biomass media, with solvents and other agents recycled within the apparatus between batches.

Particularly useful applications of the disclosed apparatus in the context of the global pandemic will be to produce completely biodegradable disposable surgical masks, which traditionally contain non-recyclable polyester. It will also serve many other humanitarian purposes that will enhance the ability to deal with pandemics and practice medicine without harming the environment.

Thus, according to one aspect of the present disclosure there is provided a Polyhydroxyalkanoate, PHA, fabrication apparatus, comprising: a pre-solvent reservoir; and a solvent reservoir. The apparatus is further comprised of a multi-stage sequential set of chambers divided by function.

A first stage module comprises a bioreactor for fermentation having a media inlet for receiving media and a biomass outlet, the biomass outlet comprising a controller-operated valve for passing fermented biomass out of the first chamber through a first filtration system.

A second stage module comprises a first controller-operated mixing chamber having a rotational element for mixing a fermented biomass with a pre-solvent and one or more heating elements for extraction and comprising a biomass inlet connected to the controller-operated valve, a pre-solvent agent inlet/outlet connected to the pre-solvent reservoir, the chamber is perforated for drainage.

A third stage module comprises a second controller-operated mixing chamber for mixing the fermented biomass with a solvent and comprising controller-operated heating element, a biomass inlet connected to the first mixing chamber, a solvent agent inlet/outlet connected to the solvent reservoir for completing the precipitation process and forming resins.

A fourth stage module comprises a controller-operated pump for collecting and passing precipitated PHA biomass from the third mixing chamber onwards.

A fifth stage module comprises a two-layer oven, the two-layer oven comprising a top layer configured to mix precipitated PHA biomass received from the fourth stage module with one or more additives to help form the physiomechanical properties needed for the final intent use of the bioplastic and a bottom layer comprising a mold for forming one or more PHA products from the mixed precipitate.

The first, second, third, fourth, and fifth stage modules may in some examples be arranged in descending order in vertical alignment with one another within a protective casing. Furthermore, a controller and connected display and interface are provided for controlling the controller-operated elements.

In some embodiments, the bioreactor is formed of a glass container.

In some embodiments, the first stage module further comprises a connection to the bioreactor for one or more sensors connected to the controller, including one or more of a pH sensor, a dissolved oxygen sensor, a carbon dioxide sensor, a nitrogen sensor, and a phosphate sensor.

The apparatus may further comprise an acid/base reservoir and controller-operated pump for controlling the one or more gas sensors. The display may further be configured to show readings from the one or more sensors.

In some embodiments, the outlet of the bioreactor is provided with a cellulose membrane filter for filtering biomass residuals.

In some embodiments, the bioreactor is provided with a valve-operated drainage outlet for cleaning.

In some embodiments, the bioreactor interior comprises one or more mixing impellers.

In some embodiments, the bioreactor is provided with an aeration tube.

In some embodiments, the pre-solvent agent is an alcohol agent that is insoluble in PHA. The alcohol agent may be a methanol or an ethanol such as Acetone.

In some embodiments, the solvent is a precipitating agent which is soluble in PHA, the solvent is chloroform.

In some embodiments, the heating element is configured to heat the biomass and solvent mixture to 60 degrees Celsius to evaporate the solvent and to facilitate recycling of the solvent bank to its tank.

In some embodiments, pre-solvent is recycled to the pre-solvent chamber from the first mixing chamber after each batch of PHA products is fabricated.

In some embodiments, the solvent is recycled to the solvent chamber from the first mixing chamber after each batch of PHA products is fabricated.

In some embodiments, the one or more additives comprise a starch additive for adding structural strength and altering the physiomechanical properties needed for the final intent use of the bioplastic to fabricated PHA products after mixing at 180 degrees Celsius.

In some embodiments, the mold of the oven is configured to produce PHA plates.

In some embodiments, the oven is configured to achieve a temperature of 180 degrees Celsius.

In some embodiments, the fifth stage module further comprises a controller-operated UV sanitising device.

In some embodiments, the apparatus further comprises a cooling fan system configured to contain the heat from the oven.

In some embodiments, casing comprises a door mechanism for accessing the two-layer split oven.

In some embodiments, the protective casing comprises a sliding-door mechanism along the full height of the apparatus for accessing the internal modules.

In some embodiments, the interface comprises a touch screen interface for controlling the one or more pumps and valves of the apparatus.

### BRIEF DESCRIPTION OF THE DRAWINGS

Various embodiments of the invention are disclosed in the following detailed description and accompanying drawings.

FIG. 1 illustrates an isometric cutaway view of an example configuration of a PHA fabrication apparatus according to the present disclosure to show the internal construction.

FIG. 2 illustrates a second isometric view of the example configuration of the PHA fabrication apparatus according to the present disclosure with exterior protective casing, door mechanisms, and solvent and pre-solvent reservoirs shown.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

The terminology used herein is for the purpose of describing particular embodiments only and is not intended to be limiting of the invention. As used herein, the term “and/or” includes any combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

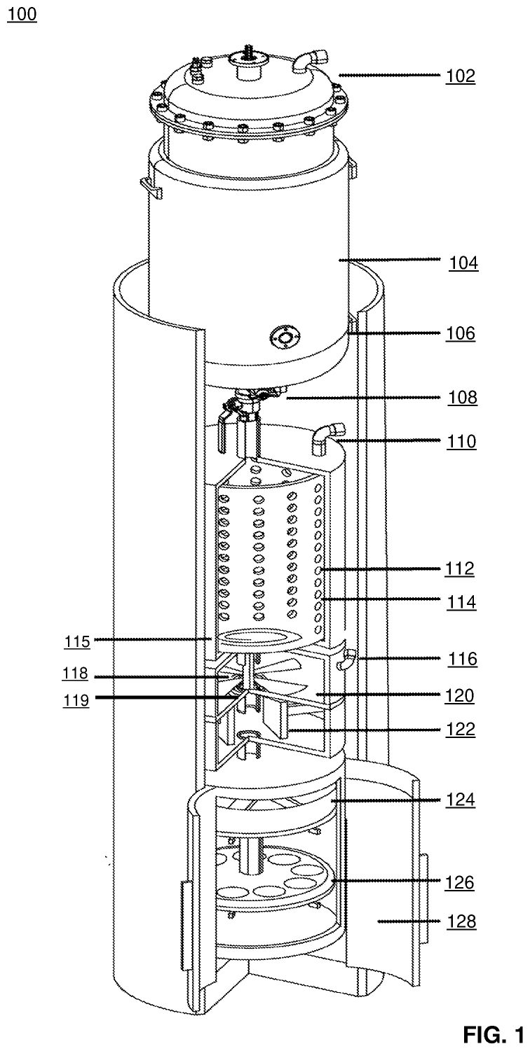

Referring to FIG. 1, an isometric cutaway view of an example configuration of a PHA fabrication apparatus according to the present disclosure is shown with the internal construction exposed.

### LIST OF REFERENCE NUMERALS

100: Bioplastic fabrication apparatus

102: Acid/base reservoir and pump system for gas sensors

104: First stage module bioreactor

106: Bioreactor Filtration system for the filtration stage

108: Biomass valve from first stage module to second stage module

110: Second stage module pre-solvent inlet/outlet

112: Second stage module washing/drying chamber for the extraction stage

114: Drainage holes for pre-solvent

115: Second stage module heating element for pre-solvent evaporation

116: Third stage module solvent inlet/outlet

118: Third stage module mixing rotor

119: Third stage module heating element for solvent evaporation

120: Third stage module PHA extraction chamber and mixing impellers for the precipitation stage

122: Fourth stage module PHA precipitate pump

124: Fifth stage module oven top-layer for mixing additives

126: Fifth stage module bottom layer plate mold

128: Oven access doors for cooling and removal of PHA products

As can be seen, the system is constructed of several different process stage chambers aligned vertically on top of one another so that the flow of fluid from top to bottom is assisted by gravity to reduce energy requirements.

The first stage of the process is carried out in a a glass bioreactor surrounded by a stainless-steel shield. Glass is used because it is easier to clean, cheap, and has high resistance to wear and tear, temperature, and acidity. There are two supporting mixing impellers inside the bioreactor.

The bioreactor is sealed but has controlled inlets and outlets including one for pouring the media and the carbon source into the bioreactor and one to allow aeration. A drainage hole may also be provided for easier cleaning.

In some examples, the bioreactor also comprises a washable and disposable cellulose membrane filter at the outlet to filter the desirable biomass from residuals.

A set of pH and gas sensors ((DO) dissolved Oxygen, CO2, Nitrogen and phosphate) monitor the reaction within the bioreactor and are fed/controlled by an acid/base pump reservoir on top of the device.

Moving downwards through the apparatus, the fermented biomass containing the PHA bioplastic is passed through a controller operated valve to a second stage mixing chamber where it is mixed with pre-solvent which is insoluble in PHA, such as an alcohol agent like ethanol, or methanol Acetone. This begins the extraction of the PHA and lyses the cells. This extraction/mixing process increases the PHA yield to a higher molecular weight.

The second stage module comprises a mixing chamber configured to vigorously rotate the biomass with the agent similarly to a slow centrifuge for a few minutes and then drain the excess fluid through the illustrated holes. The pre-solvent evaporates to a separate pre-solvent reservoir tank to be recycled for the next batch.

The second stage module also includes heating elements which begin heating the mixture up to 60 Celsius degrees to evaporate the pre-solvent and separate it from the PHA. The pre-solvent evaporates and is collected back to a pre-solvent reservoir tank to be recycled for use in the next batch.

When the mixing and draining of the second stage is complete, the formed mass will be collected in a third stage precipitation chamber and mixed with a solvent which is soluble in PHA such as chloroform. The solvent chamber here mixes the PHA with the chloroform to precipitate a high molecular weight of PHA.

The third stage also includes heating elements which begin heating the mixture up to 60 Celsius degrees to evaporate the solvent and separate it from the PHA. The solvent evaporates and gets collected back to a pre-solvent reservoir tank to be recycled for use in the next batch.

The fourth stage chamber is where the actual PHA plastic gets collected and pumped onwards into the fifth stage where there is a two-layer oven comprising a mold.

The PHA gets pumped into the first oven's top layer and in the present example is mixed with starch as an additive to provide additional structural strength to the PHA bioplastic plates. After mixing the PHA/starch mixture is pumped downwards again into the oven's bottom layer for the mold to form the plates—or whichever other mold is being used to from the desired PHA product. The oven temperature may be 180 degrees Celsius. A UV sanitising light may be provided to sanitize the oven in between batches. Furthermore, a cooling system may be provided in the from of a supportive fan. The oven has a pair of access doors which allow a user to withdraw molds and products from the oven.

The fifth stage heating of the PHA granules in the lower compartment of the oven is the final step and will result in freshly made PHA products, in this case a set of plates because a plate mold was used.

Thus the integrated apparatus provides for down-streaming of the fabrication process from fermentation in the first stage, filtration and extraction in the second stage, precipitation, mixing, and evaporation in the third stage, pumping forwards and injecting in the fourth stage, and heating and drying the granules of the bioplastics Polyhydroxyalkanoate (PHA) plastic inside the desired mold in the fifth stage. This integrated process flow is able to produce plastic plates, or whatever other PHA product is desired based on the mold used in the oven in the fifth stage, in a single “batch”.

Furthermore, the whole process can be controlled and monitored by a touchscreen interface connected to a controller of the device such as a microprocessor which operates all the relevant valves, pumps, sensors, and other electrical components.

Thus the apparatus can also include a digital screen such as a touchscreen interface to control operations such as the injection of the plastic into the mold, to set the pressurized pump in the third compartment, and to monitor the flow of the inlets and outlets of the bioreactor to maintain the DO dissolved oxygen levels and other necessary parameters.

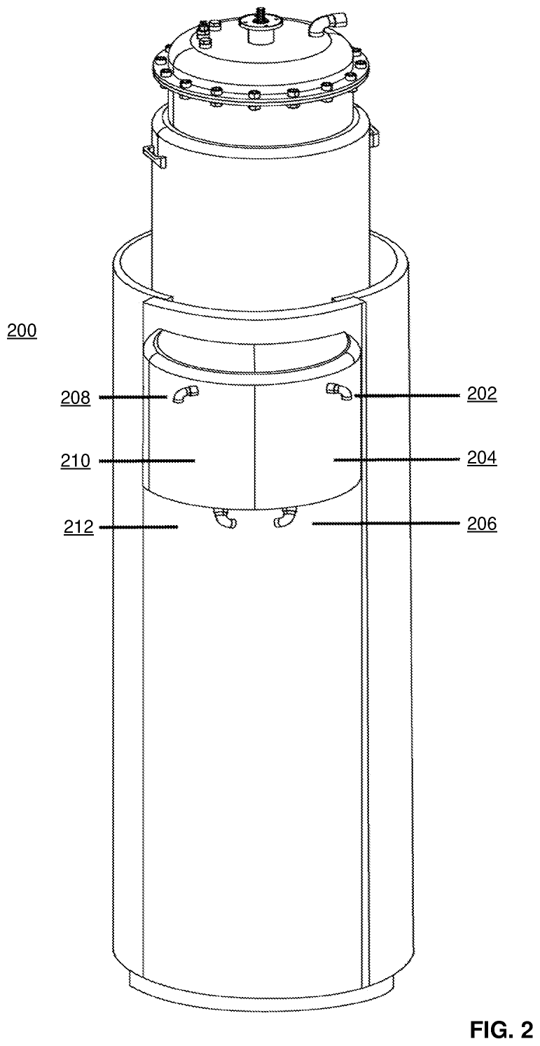

Referring to FIG. 2, a second isometric view of the example configuration of the PHA fabrication apparatus according to the present disclosure is shown with the exterior protective casing, door mechanisms, and solvent and pre-solvent reservoirs shown.

### LIST OF REFERENCE NUMERALS

200: Reservoir chambers

202: Pre-solvent reservoir inlet for evaporated agent

204: Pre-solvent reservoir tank

206: Pre-solvent reservoir outlet for liquid agent

208: Solvent reservoir inlet for evaporated agent

210: Solvent reservoir tank

212: Solvent reservoir outlet for liquid agent

As can be seen, a protective circular casing provides shield around the entire device that is divided into three parts. A larger part that encompasses the device and two smaller sliding parts that function as a slide-door mechanism for allowing access to the device interior. Of course, other configurations may also be used.

The set of reservoir tanks with inlets and outlets to the various internal process stages are also shown mounted to the sliding door part.

While fermentation-based bioplastic fabrication is the most environmentally friendly method of manufacture, there are disadvantages in terms of the limitations on the speed of the process.

Even with the integrated design disclosed herein, the bacterial extraction and injection processes from the bioreactor can take up to two days. For example, research results using currently available technology have yielded results of 12 hours for the growth of bacteria, which is followed by the start of PHA production at a rate of approximately 1 g PHA/L/hr. At these rates, in 24 hours up to 83 g/L biomass and 24 g/L PHA is typically produced. If the time for the extraction and precipitation steps and the drying of the mold are also taken into account the entire process takes up to 48 hours. Other bioplastics, for example those that are fabricated directly from plants, do not take so long because there is no need to generate biomass first.

Other common issues with bioplastics are also still present, such as the limited shelf life of 18 months at room temperature.

These disadvantages can however be overcome in future developments using recombinant DNA technologies.

The bacteria strains used can be modified for a higher plastic yield, and for specific metabolism pathways that could produce a higher-grade plastic for faster and easier logistics. The expiry date can be enhanced.

Some types of bioplastics have a reputation of being more brittle with high degree of crystallinity due to high melting point and low degradation temperature, making it difficult to thermally process. This can be overcome by producing a medium chain length polymer.

The major current applications of the device are to make cups, plates, utensils, toys, bags, packaging, masks, however the scope of possible products that could be manufactured is much broader.

Biomaterials for the biomedical applications such as grafts, implants, scaffold, sutures, as well as cosmetic products such as exfoliants, skin rejuvenation creams, and the make-up industry may also be fabricated with minor modifications to the fabrication process and minimal adjustments to the apparatus configuration disclosed herein.

For example, for medical product fabrication, additional purification steps would be required subsequent to the oven-mold stage, and alterations to re reagents and additives used would be made according to the type of end product desired. A high-yield non-pathogenic bacteria strain capable of passing the toxicity test and ensuring total biocompatibility with the human body would also need to be developed.

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the bioplastic fabrication apparatus have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A Polyhydroxyalkanoate, PHA, fabrication apparatus, comprising:
a pre-solvent reservoir;
a solvent reservoir;
a first stage module comprising a bioreactor for fermentation having a media inlet for receiving media and a biomass outlet, the biomass outlet comprising a controller-operated valve for passing fermented biomass out of the first chamber through a first filtration system;
a second stage module comprising a first controller-operated mixing chamber having a rotational element for mixing a fermented biomass with a pre-solvent and one or more heating elements for extraction, and comprising a biomass inlet connected to the controller-operated valve, a pre-solvent agent inlet/outlet connected to the pre-solvent reservoir, the chamber is perforated for drainage;
a third stage module comprising a second controller-operated mixing chamber for mixing the fermented biomass with a solvent and comprising controller-operated heating element, a biomass inlet connected to the first mixing chamber, a solvent agent inlet/outlet connected to the solvent reservoir for completing the precipitation process and forming resins;
a fourth stage module comprising a controller-operated pump for collecting and passing precipitated PHA biomass from the third mixing chamber onwards;
a fifth stage module comprising a two-layer oven, the two-layer oven comprising a top layer configured to mix precipitated PHA biomass received from the fourth stage module with one or more additives to help form the physiomechanical properties needed for the final intent use of the bioplastic and a bottom layer comprising a mold for forming one or more PHA products from the mixed precipitate;
a protective casing, wherein the first, second, third, fourth, and fifth stage modules are arranged in descending order in vertical alignment with one another within the casing; and
a controller and connected display and interface for controlling the controller-operated elements.

2. A PHA fabrication apparatus according to claim 1, wherein the bioreactor is formed of a glass container.

3. A PHA fabrication apparatus according to claim 1, wherein the first stage module further comprises a connection to the bioreactor for one or more sensors connected to the controller, including one or more of a pH sensor, a dissolved oxygen sensor, a carbon dioxide sensor, a nitrogen sensor, and a phosphate sensor.

4. A PHA fabrication apparatus according to claim 3, wherein the apparatus further comprises an acid/base reservoir and controller-operated pump for controlling the one or more gas sensors.

5. A PHA fabrication apparatus according to claim 3, wherein the display is further configured to show readings from the one or more sensors.

6. A PHA fabrication apparatus according to claim 1, wherein the outlet of the bioreactor is provided with a cellulose membrane filter for filtering biomass residuals.

7. A PHA fabrication apparatus according to claim 1, wherein the bioreactor is provided with a valve-operated drainage outlet for cleaning.

8. A PHA fabrication apparatus according to claim 1, wherein the bioreactor interior comprises one or more mixing impellers.

9. A PHA fabrication apparatus according to claim 1, wherein the bioreactor is provided with an aeration tube.

10. A PHA fabrication apparatus according to claim 1, wherein the pre-solvent agent is an alcohol agent that is insoluble in PHA.

11. A PHA fabrication apparatus according to claim 10, wherein the alcohol agent is a methanol or an ethanol such as Acetone.

12. A PHA fabrication apparatus according to claim 1, wherein the solvent is a precipitating agent which is soluble in PHA.

13. A PHA fabrication apparatus according to claim 12, wherein the solvent is chloroform.

14. A PHA fabrication apparatus according to claim 1, wherein the heating element is configured to heat the biomass and solvent mixture to 60 degrees Celsius to evaporate the solvent and to facilitate recycling of the solvent bank to its tank.

15. A PHA fabrication apparatus according to claim 1, wherein pre-solvent is recycled to the pre-solvent chamber from the first mixing chamber after each batch of PHA products is fabricated.

16. A PHA fabrication apparatus according to claim 1, wherein solvent is recycled to the solvent chamber from the first mixing chamber after each batch of PHA products is fabricated.

17. A PHA fabrication apparatus according to claim 1, wherein the one or more and altering the physiomechanical properties needed for the final intent use of the bioplastic to fabricated PHA products after mixing at 180 degrees Celsius.

18. A PHA fabrication apparatus according to claim 1, wherein the mold of the oven is configured to produce PHA plates.

19. A PHA fabrication apparatus according to claim 1, wherein the oven is configured to achieve a temperature of 180 degrees Celsius.

20. A PHA fabrication apparatus according to claim 1, wherein the fifth stage module further comprises a controller-operated UV sanitising device.

21. A PHA fabrication apparatus according to claim 1, wherein the apparatus further comprises a cooling fan system configured to contain the heat from the oven.

22. A PHA fabrication apparatus according to claim 1, wherein casing comprises a door mechanism for accessing the two-layer split oven.

23. A PHA fabrication apparatus according to claim 1, wherein the protective casing comprises a sliding-door mechanism along the full height of the apparatus for accessing the internal modules.

24. A PHA fabrication apparatus according to claim 1, wherein the interface comprises a touch screen interface for controlling the one or more pumps and valves of the apparatus.
